(19) United States
(12) Patent Application Publication
(10) Pub. No.: US 2015/0320597 A1
(43) Pub. Date: Nov. 12, 2015

(54) Lens Nucleus Chopper

(71) Applicant: Sergio Jacobovitz, Ville de Montagne (BR)

(72) Inventor: Sergio Jacobovitz, Ville de Montagne (BR)

(21) Appl. No.: 14/709,313

(22) Filed: May 11, 2015

Related U.S. Application Data
(60) Provisional application No. 61/990,805, filed on May 9, 2014.

(51) Int. Cl. A61F 9/007

(52) U.S. Cl. CPC A61F 9/00754; A61F 9/00745

(57) ABSTRACT

A cataract surgery apparatus for fragmenting an eye lens nucleus including a retractor surface for retracting a capsular bag of an eye, a knife edge for chopping the lens nucleus and longitudinal sides for splitting the chopped lens nucleus. The cataract surgery apparatus fragments an eye lens with a bimanual operation of two cataract surgery apparatus. A single cataract surgery apparatus can also fragment a target eye lens nucleus.

### CROSS-REFERENCE TO RELATED APPLICATIONS

This application claims the benefit of U.S. Provisional Application Ser. No. 61/990,805, filed on May 9, 2014, entitled Medical Instrument For Cataract Surgery, this prior application is herewith incorporated by reference in its entirety.

A lens nucleus is a transparent biconvex lens that refracts and transmits light to a retina of an eye. As such, a healthy lens nucleus is crucial for clear eyesight. An eye lens is located behind an iris of an eye, and enclosed within an elastic capsular bag. However, due to age and/or disease, a lens may become opaque or cloudy, resulting in a condition known as a cataract. A cataract may severely impair vision, and may require cataract surgery.

### BACKGROUND OF THE INVENTION

1. Field of the Invention

The present invention relates to ophthalmology, and more particularly to a cataract surgery apparatus for chopping a lens nucleus of an eye.

2. Description of the Related Art

Cataract surgery usually includes extracting a diseased lens nucleus and implanting a functioning lens, such as an artificial lens nucleus implant.

Common techniques for removing a cataract may include first removing a portion of a capsular bag (i.e. capsulorhexis) and extracting the lens nucleus via manual and/or automated tools.

A diseased lens nucleus may be extracted in various ways. As a first example, a diseased lens nucleus may be removed via a phacoemulsifier. A phacoemulsifier is a device typically used in modern cataract surgery, for emulsifying and aspirating a diseased lens. Usually, a phacoemulsifier emulsifies a diseased lens nucleus by delivering ultrasonic energy to the lens nucleus via a hollow tip that oscillates at an ultrasonic frequency, and aspirates emulsified particles. However, such ultrasonic energy delivered by a phacoemulsifier may damage other tissues in a vicinity of a target lens nucleus. For example, the endothelium of an eye is a delicate structure that may be irreversibly damaged by energy and/or forces delivered by a phacoemulsifier. The corneal endothelium is responsible for maintaining corneal transparency; once damaged it could lead to a corneal edema and if severely damaged a corneal transplant may be required. As such, it is important to minimize the amount of ultrasonic energy delivered to surrounding tissues. Typically, phacoemulsification may be aided by fragmenting a diseased lens nucleus. For example, a surgeon may attempt to chop or fragment a diseased lens nucleus before or during phacoemulsification, such that the diseased lens nucleus may be more easily removed, subsequently reducing exposure time to damaging ultrasonic energy.

As a second example, a diseased lens nucleus may be manually removed through a corneal incision near a scleral area of an eye, without a phacoemulsifier. A diseased lens may be manually removed via a lens loop apparatus, a spatula, forceps, or the like.

Manual methods may be chosen for various reasons. For example, manual methods may be more appropriate in less developed countries where phacoemulsification techniques are too expensive. Further, manual methods may be used to extract a lens nucleus for which a phacoemulsifier may not be effective.

In manual methods, an incision is usually appropriately sized to remove a diseased lens nucleus. However, a larger sized incision may cause greater damage and inflammation to eye tissues, longer recovery times, and post operation complications, compared to smaller sized incisions. A smaller incision is usually desired for manually removing cataracts. Therefore, manual techniques sometimes include chopping a diseased lens nucleus into fragments before manually removing the diseased lens nucleus, thus requiring a smaller incision.

As described above, chopping or fragmenting a nucleus is useful for both phacoemulsification methods and manual extraction methods. However, traditional chopping apparatuses and methods pose various problems, disadvantages and/or limitations.

For example, one chopping method involves first using a phacoemulsifier to apply a groove in a lens nucleus, rotating the lens nucleus 90 degrees, and applying a second groove. Then, the lens nucleus may be split into four pieces by a separate cracking device, otherwise known as a nucleus splitter. Alternatively this method may involve applying only one groove. Nevertheless, such a groove method may deliver substantial ultrasonic energy to tissues in the vicinity of a target lens, and may be considered ineffective for harder cataract conditions. Further, this method requires multiple tools to chop or fragment a diseased lens nucleus, increasing operation time and trauma to an eye.

Commonly, chopping a lens nucleus may be attempted via various types of chopper apparatuses. For example, one traditional chopper apparatus includes a handle with an L-shaped tip that is bent away from the handle, where the tip is used to apply a plunge-force into a target lens nucleus. Such a traditional chopper may include a sharp edge at a side of the tip near the handle, and is commonly used bimanually with a phacoemulsifier, for attempting to chop a lens nucleus. These two configurations can be seen in the incorporated references, “Phaco Chop Techniques—Comparing Horizontal vs. Vertical Chop” (Chang), U.S. Patent Application No. 2003/0093099 filed by Anthone, and U.S. Pat. No. 8,974,480 issued to Terao. However, such a chopper has various disadvantages and limitations. For example, such traditional choppers require application of a vertical force that is normal to an equatorial plane of a lens nucleus to apply a plunge-cut. Such vertical forces may cause a posterior portion of a capsular bag to rupture or tear, or cause severe damage to zonular fibers and endothelium areas. Further, such a traditional chopper can only apply a chopping depth that is limited by its handle. Even further, such a tip configured for plunge-cutting may cause the handle to contact other sensitive areas, such as corneal endothelium areas.

Such a traditional chopper may also include a sharp edge disposed at a posterior portion of the chopper tip, toward the handle, for applying a horizontal chopping action when the apparatus is pulled in a direction toward the handle. However, this configuration is problematic because the tip must first be inserted toward a central chopping point before engaging a lens nucleus. If a traditional chopper tip is misplaced upon engaging a lens nucleus, the traditional chopper may directly engage zonular fibers, causing irreversible damage. Such a traditional chopper requires at least two substantial motions for attempting to chop or fragment a lens nucleus, causing unnecessary and undesired trauma to incision points, increased surgery time, and increased chance of contamination or infection. Further, one of the two motions required by a traditional chopper does not provide any chopping action. As described above, increased cataract surgery time during phacoemulsification may cause increased damage to an eye. Further, when used during phacoemulsification, such a traditional chopper is applied by dragging the apparatus across the lens nucleus toward the handle or entrance point at an incision, and does not optimally deliver vector forces to aid phacoemulsification. For example, a traditional chopper having a sharp edge near a handle as described above provides vector forces to a diseased lens that may not appropriately cancel or neutralize vector forces applied by a phacoemulsifier tip, since horizontal forces applied by a traditional chopper are towards an incision point of the tradition chopper, and a phacoemulsifier cannot be inserted through the same incision point. For example, a traditional chopper is not able to use its sharp edge to chop a lens nucleus while a phacoemulsifier tip is pushed toward the lens nucleus without unwanted rotation of the lens nucleus. Such a traditional bladed chopper “pulls” rather than “pushes” a lens nucleus, which may cause the lens nucleus to undesirably rotate, since a traditional chopper handle is limited by a position of a phacoemulsifier handle, and is usually inserted via an incision point near an incision point for a phacoemulsifier. For example, a traditional chopper may be inserted near an incision of a phacoemulsifier such that the traditional chopper “pulls” the lens nucleus toward a tip of the phacoemulsifier, to concurrently chop and deliver lens fragments to the phacoemulsifier tip, which causes discomfort during bimanual operation of the two tools, and may rotate, instead of fragment, the target lens nucleus. Bimanual operation via incisions placed 180 degrees apart relative to a lens nucleus center is desirable for manual convenience. However, as described above, a traditional chopper “pulls” rather than “pushes” with a sharp edge, and inserting a traditional chopper 180 degrees apart from a phacoemulsifier incision does not allow a traditional chopper to appropriately chop during phacoemulsification, and in this case, the traditional chopper will merely horizontally shift or move a lens nucleus, applying forces to sensitive areas surrounding the lens nucleus since the phacoemulsifier does not provide proper counter forces to neutralize a net horizontal force applied to the lens nucleus. Additionally, during phacoemulsification, a traditional chopper tip may be blocked by a phacoemulsifier, since a traditional chopper is usually inserted near an insertion point of a phacoemulsifier such that the chopper may “pull” a diseased lens nucleus toward the phacoemulsifier tip. As such, engagement area of a traditional chopper tip may be severely limited when used in conjunction with a phacoemulsifier tool.

When a phacoemulsifier is not being used, a traditional chopper has no means to cancel horizontal chopping vector forces. As such, it is desirable to hold, grip or apply horizontal forces to a lens nucleus such that horizontal chopping forces are neutralized or cancelled. As such, it is desirable to first retract a capsular bag rim (after a capsulorhexis procedure) of an eye, to engage and provide forces to a target nucleus lens such that horizontal chopping forces may be neutralized.

Another traditional chopper apparatus includes a cross-action forcep structure with sharp paddles on each tip of the forceps. This is also known as an Akahoshi pre-chopper, and can be seen discussed in the incorporated reference, U.S. Pat. No. 8,974,480 issued to Terao. This chopper suffers from similar problems of other traditional choppers. For example, an Akahoshi pre-chopper applies substantial vertical force to a lens nucleus, and may not properly chop or fragment hard cataract conditions. Further, in soft cataract conditions, an Akahoshi pre-chopper may merely “mash” contact portions without fully splitting or fragmenting a diseased nucleus. Therefore, an Akahoshi pre-chopper is mainly useful for intermediate cataract densities. The Akahoshi apparatus may be problematic for a shallower anterior chamber of the eye as its large dimensions could pose risks to the corneal endothelium layer.

As such, there exists a need for a lens nucleus chopping apparatus that:

enables a surgeon to apply appropriate vector forces in a horizontal plane (e.g. equatorial plane) of a lens nucleus during chopping maneuvers;

enables sequential retracting of the capsular bag and chopping of the lens nucleus;

allows convenient maneuvering of a chopping tip such that a handle or tip of the chopping apparatus is less limited by a phacoemulsifier or other tool during bimanual operation;

conveniently and sequentially allows cracking, splitting or fracturing a lens nucleus after the lens nucleus has been substantially chopped;

enables chopping of a wide range cataract densities (e.g. soft, intermediate, hard); and

enables improved chopping of a diseased lens nucleus without phacoemulsification.

### SUMMARY OF THE INVENTION

It is accordingly an object of the invention to provide a lens nucleus chopper apparatus and method for cataract surgery which overcome the above-mentioned disadvantages of the heretofore-known devices and methods of this general type.

With the foregoing and other objects in view there is provided, an eye lens dividing apparatus comprising two instruments. Each instrument of the two instruments includes a handle with a longitudinal end, a shaft extending from the longitudinal end to a terminal end of the shaft, a multifunctional tip disposed at the terminal end, the multifunctional tip having a knife edge, a retractor surface opposite the knife edge, and lateral surfaces opposite one another and extending from the knife edge to the retractor surface. The knife edge extends from the terminal end and faces in substantially a same direction as the terminal end.

In accordance with an added feature of the invention, edges of the lateral surfaces define a bottom surface from the knife edge to the retractor surface.

In accordance with an added feature of the invention, the bottom surface and the retractor surface have a smooth finish.

In accordance with an additional feature of the invention, the bottom surface and the retractor surface have a rounded cross section.

In accordance with yet an additional feature of the invention, the rounded cross section is defined by a full radius between the lateral surfaces.

In accordance with yet another added feature of the invention, the rounded cross section is defined by a two corner radii at corners of the lateral surfaces.

In accordance with still another added feature of the invention, the bottom surface is defined by a curve.

In accordance with a further additional feature of the invention, the knife edge has a concave extent.

In accordance with a further added feature of the invention, each retractor surface is configured to retract a capsular bag of an eye lens.

### BRIEF DESCRIPTION OF THE DRAWINGS

FIG. 1 is a perspective view of a first embodiment of a lens nucleus chopper;

FIG. 2 is an enlarged perspective view of detail “A” in FIG. 1 showing a tip of the lens nucleus chopper;

FIG. 3A is a side view of the tip of the lens nucleus chopper;

FIG. 3B is a cross-sectional view A of the tip of the lens nucleus chopper, the cross section taken along sectional plane A-A indicated in FIG. 3A;

FIG. 3C is a cross-sectional view B of the tip of the lens nucleus chopper, the cross section taken along sectional plane B-B indicated in FIG. 3A;

FIG. 3D is a cross-sectional view C of the tip of the lens nucleus chopper, the cross section taken along sectional plane C-C indicated in FIG. 3A;

FIG. 3A′ is a side view of a second embodiment of a nucleus chopper tip;

FIG. 3B′ is a cross-sectional view A′ of the second embodiment of the lens nucleus chopper tip, the cross section taken along sectional plane A′-A′ indicated in FIG. 3A′;

FIG. 3C′ is a cross-sectional view B′ of the second embodiment of the lens nucleus chopper tip, the cross section taken along sectional plane B′-B′ indicated in FIG. 3A′;

FIG. 3D′ is a cross-sectional view C′ of the second embodiment of the lens nucleus chopper tip, the cross section taken along sectional plane C′-C′ indicated in FIG. 3A′;

FIG. 4 is a perspective view showing a pair of lens nucleus choppers with the lens nucleus choppers in a first position prior to chopping a lens nucleus;

FIG. 5 is a perspective view of the pair of lens nucleus choppers in a second position after horizontally chopping the lens nucleus;

FIG. 6 is a plan view of a lens nucleus after a capsulorhexis procedure has been performed prior to chopping;

FIG. 7 is a plan view of the lens nucleus, showing a pair of lens nucleus choppers retracting a capsular bag of the lens nucleus;

FIG. 8 is a plan view of the lens nucleus, showing a pair of lens nucleus choppers horizontally chopping the lens nucleus;

FIG. 9 is a plan view of the lens nucleus, showing a pair of lens nucleus choppers at the onset of cracking the lens nucleus after substantially horizontally chopping the lens nucleus;

FIG. 10 is a plan view of the lens nucleus, showing a pair of lens nucleus choppers having cracked the lens nucleus;

FIG. 11 is a plan view of the lens nucleus, showing a rotation of the lens nucleus after chopping and cracking the lens nucleus;

FIG. 12 is a plan view of the lens nucleus, showing horizontally chopping and cracking a half of the rotated lens nucleus of FIG. 11;

FIG. 13 is a plan view of the lens nucleus of FIG. 12, showing a phacoemulsifier emulsifying and aspirating the lens nucleus;

FIG. 14 is a plan view of the lens nucleus of FIG. 13, showing a lens nucleus chopper chopping the lens nucleus during phacoemulsification;

FIG. 15A is a side view of a lens nucleus in an eye, showing an equatorial plane in dashed line and lens nucleus choppers retracting a capsular bag rim;

FIG. 15B is a cross-sectional view of an eye, showing a lens nucleus and surrounding tissue;

FIG. 16A is a side view of a third embodiment of a lens nucleus chopper tip;

FIG. 16B is a side view of a fourth embodiment of a lens nucleus chopper tip;

FIG. 16C is a side view of a fifth embodiment of a lens nucleus chopper tip; and

FIG. 16D is a side view of a sixth embodiment of a lens nucleus chopper tip.

It is to be understood that in the drawings, like reference numbers indicate like elements.

### DESCRIPTION OF THE PREFERRED EMBODIMENTS

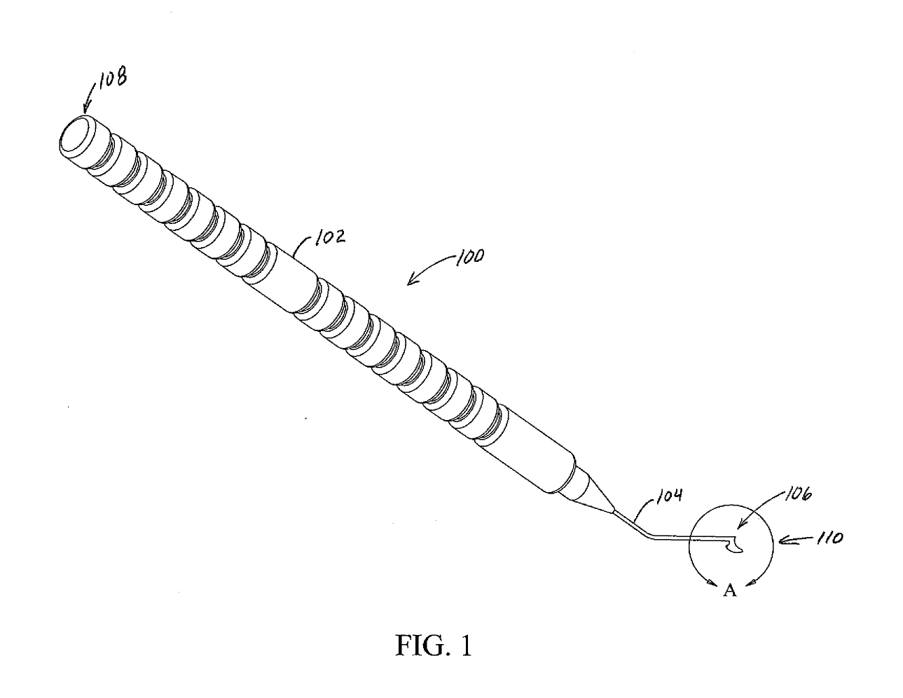

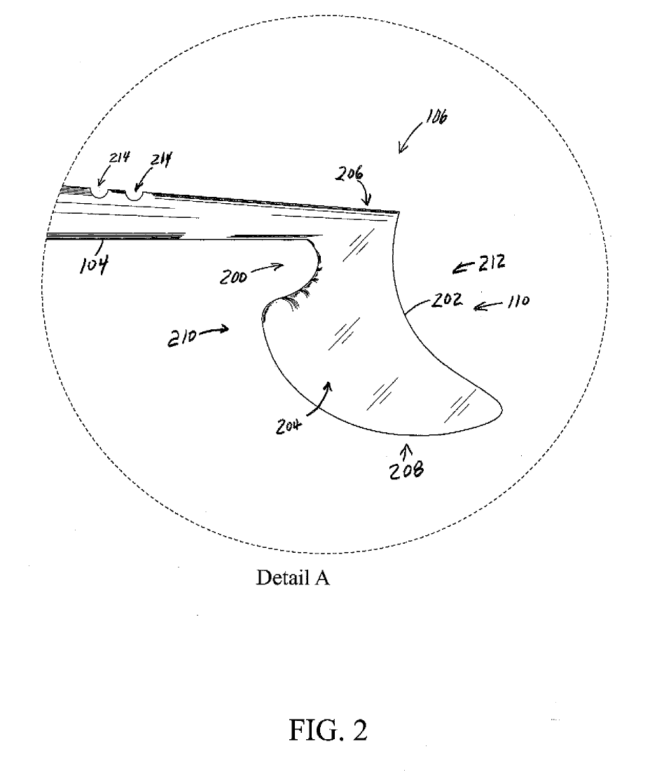

FIG. 1 shows a first embodiment of a lens nucleus chopping apparatus. Particularly, FIG. 1 shows nucleus chopper 100 including a handle 102, a shaft 104 and a multifunctional tip 106. Multifunctional tip 106 is connected to shaft 104 at a terminal end of shaft 104. Shaft 104 is connected to handle 102. For purposes of description, handle 102 is disposed at a posterior end 108 of nucleus chopper 100. Multifunctional tip 106 is disposed at an anterior end 110 of nucleus chopper 100. Shaft 104 may be configured to be substantially parallel and coaxial to handle 102, or be bent at an angle relative to handle 102. For example, FIG. 1 shows shaft 104 in a bent configuration. Shaft 104 may also subtend an angle to multifunctional tip 106. Shaft 104 may subtend an angle greater than 90 degrees relative to a line drawn from top side 206 to bottom side 208 of multifunctional tip 106. Shaft 104 may also have any appropriate cross sectional profile or area. For example, shaft 104 may have an ellipsoid or round profile. Shaft 104 and multifunctional tip 106 may be configured in any appropriate way in accordance with aspects of this disclosure. For example, shaft 104 may include various dimensional reference notches, providing reference for a surgeon inserting nucleus chopper 100 into an eye. As such, shaft 104 includes reference notches 214 shown in FIG. 2. For example, reference notches 214 may provide a guide for a surgeon to determine how far multifunctional tip 106 has been inserted into a corneal lens cavity for manipulating and/or chopping a lens nucleus. The reference notches 214 may be spaced less than 1 mm apart, as a non-limiting example. Multifunctional tip 106 may have a maximum thickness of 120 micrometers.

Shaft 104, multifunctional tip 106 and handle 102 may include various materials. For example, various combinations of wood, metal, plastic, stainless steel, titanium, and/or ceramic materials may compose these components. It is to be understood that multifunctional tip 106 and/or shaft 104 or their various elements may be composed of and/or include cheap and/or disposable parts, such as plastic. Preferably, multifunctional tip 106 is a hard metal that may be used as a cutting edge for chopping, cutting and/or slicing a lens nucleus of an eye. Multifunctional tip 106 and its elements may be composed of a shared and/or continuous material. Multifunctional tip 106 may or may not be composed of a same material as shaft 104 and/or handle 102.

Multifunctional tip 106 and/or shaft 104 may be manufactured in various ways. For example, a stainless steel wire with a diameter of 0.95 mm may be sanded via sandpaper of various grit, such as 120 grit sandpaper. Further, various abrasives may be used to remove any grooves caused by the sandpaper. A distal portion of the sanded wire may be then hammered to a point. The hammered distal portion may then be shaped into a cutting edge (e.g. knife edge portion 202) via further disc sanding while viewed under a microscope. The multifunctional tip 106 and/or shaft 104 may be polished via felt polishers and polishing paste.

Multifunctional tip 106 and/or shaft 104 may be configured to be exchangeable or replaceable. For example, multifunctional tip 106 and/or shaft 104 may be removably attached to one another or handle 102, such that various different tips and/or shafts may be chosen and attached to nucleus chopper 100. Such a function may be achieved via any appropriate means, such as snap-lock mechanisms, latches or hinges. Any appropriate means may be used to removably attach different tips and/or shafts in accordance with aspects of the present invention.

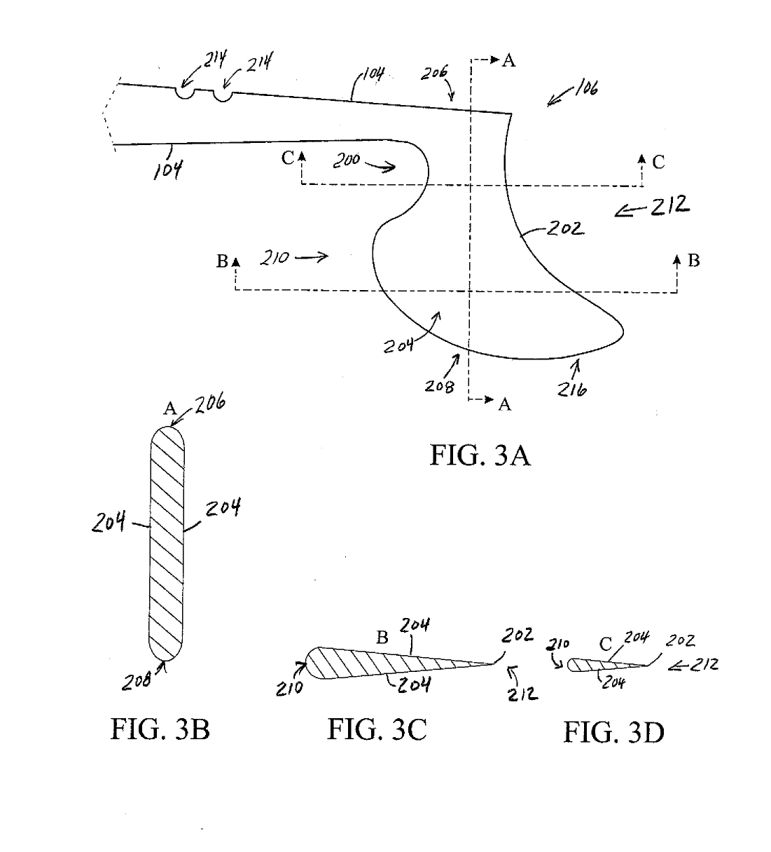

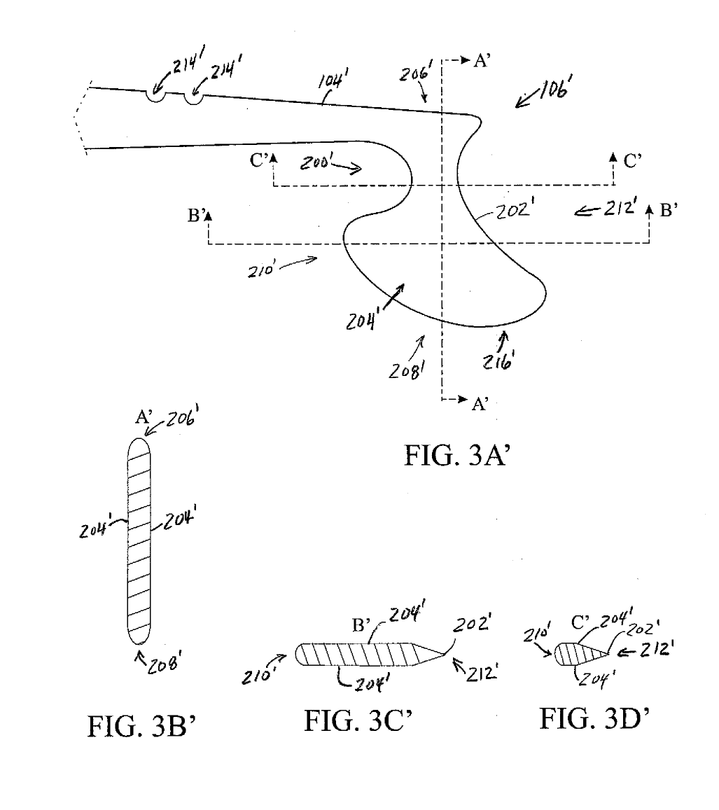

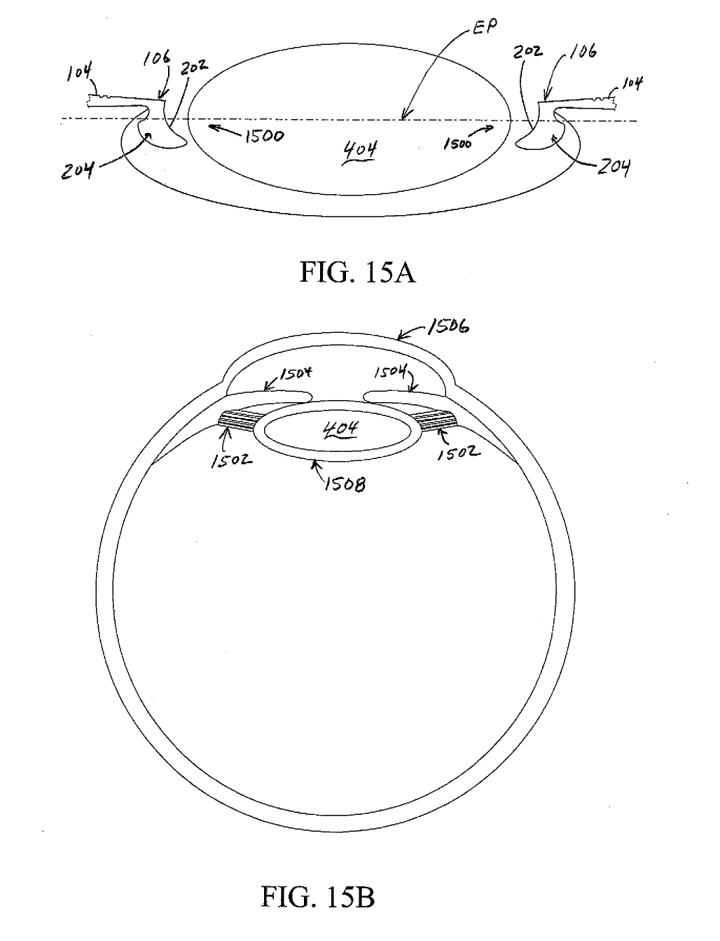

Turning to FIG. 2, multifunctional tip 106 includes a retractor portion 200, a knife edge portion 202, and lateral sides/surfaces defining cracker areas 204. Cracker areas 204 are areas formed between a top side 206, bottom side 208, an anterior tip side 212 and a posterior tip side 210 of multifunctional tip 106. For example, FIGS. 3A, 3B, and 3C, and 3D show cracker areas 204 as described above. As such, cracker areas 204 are on opposite lateral sides of multifunctional tip 106. Cracker areas 204 are configured for manipulating a lens nucleus. For example, when engaged with a lens nucleus, cracker areas 204 may allow an operator to rotate and or manipulate the lens nucleus and/or lens nucleus fragments. As such, cracker areas 204 may be configured to have a particular surface area or be composed of a particular material to deliver proper frictional and/or other forces for appropriately manipulating a lens nucleus. For example, cracker areas 204 may include a roughened finish to aid in cracking or fracturing a lens nucleus as described in detail herein. Further, areas of cracker areas 204 near bottom side 208 may be more polished compared to areas of cracker areas 204 near top side 206. For example, cracker areas 204 may be configured such that bottom side 208 is smooth and highly polished, and such that posterior portions of a capsular bag of an eye are not damaged when multifunctional tip 106 chops a nucleus as described herein. Bottom side 208 of cracker areas 204 may include a smooth surface finish, where the smooth finish is defined as a polished machine finish that allows bottom side 208 to contact and/or move along a posterior portion of a lens capsule without rupturing or tearing the posterior portion of the lens capsule in a situation when the bottom side 208 contacts a posterior area of the lens capsule during cutting of the lens nucleus. Cracker areas 204 near bottom side 208 may include a blunt protruding portion 216 for rotating a lens nucleus, or translating fragments of a lens nucleus in a horizontal direction. Further, cracker areas 204 may be configured to manipulate a lens nucleus in a horizontal and/or radial direction and/or action (i.e. forces substantially parallel to an equatorial plane of the lens nucleus) without substantial vertical forces (i.e. forces normal to an equatorial plane of the lens nucleus). For example, via bimanually operating two of nucleus choppers 100, cracker areas 204 allow a surgeon to split and/or crack a nucleus into two parts by applying horizontal action or forces as described above. The above described equatorial plane is indicated by a dashed line EP in FIG. 15A. Turning back to FIG. 2, retractor portion 200 is formed between shaft 104 and bottom side 208, where cracker areas 204 and shaft 104 substantially meet. Retractor portion 200 may take form as a curved structure disposed toward a posterior side of multifunctional tip 106. Such a posterior side is indicated as posterior tip side 210, and similarly an anterior tip side of multifunctional tip 106 is indicated as anterior tip side 212. The curved portion of retractor portion 200 is concave such that its apex is disposed toward the anterior tip side 212. As such, retractor portion 200 is shaped for retracting a capsular bag of an eye. The distal region of retractor portion 200 may have a large radius so as to minimize possible damage to the capsular bag during retraction of the capsular bag. Retractor portion 200 may also be configured to retract an iris of an eye. Cracker areas 204 and retractor portion 200 are configured to safely engage and interact with tissues of an eye without causing damage. For example, cracker areas 204 and retractor portion 200 are smooth and rounded compared to knife edge portion 202.

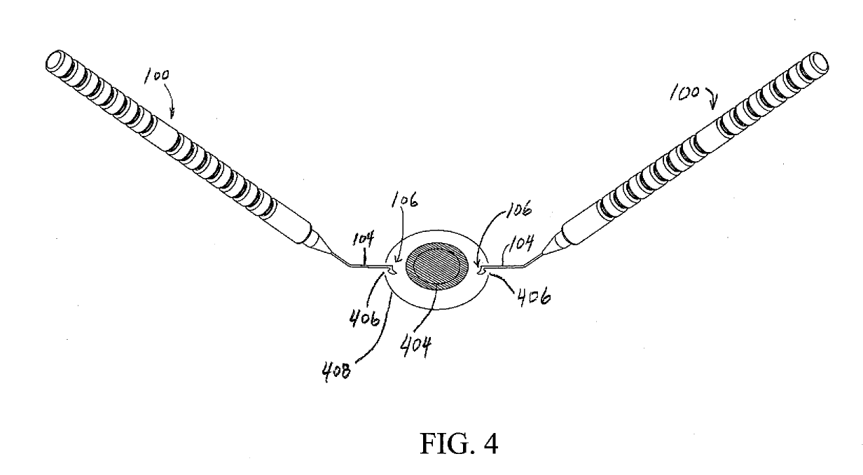

Knife edge portion 202 is a cutting edge disposed at anterior end 110 of nucleus chopper 100. Knife edge portion 202 extends from a terminal end of the shaft, from top side 206 to bottom side 208 of multifunctional tip 106. Knife edge portion 202 is configured to cut, slice, and or chop a lens nucleus when an operator applies action toward anterior end 110 by maneuvering handle 102. Such action is further described below with respect to FIGS. 4-12 and 14. Retractor portion 200 allows an operator to retract a capsular bag of an eye for placing knife edge portion 202 on an equatorial portion 1500 of a lens capsule, as shown in FIG. 15A. As such, an operator may sequentially maneuver handle 102 so that knife edge portion 202 chops and/or cuts a lens nucleus in a direction toward anterior end 110 sequentially after retracting a capsular bag. Additionally, it is to be understood that an operator may adjust a cutting and/or chopping direction at will during cutting and/or chopping. For example, while pushing knife edge portion 202 for chopping a lens as described above, an operator may adjust a chopping plane in a radial and/or horizontal direction relative to an equatorial plane of a lens nucleus. Additionally, an operator may adjust a chopping plane of knife edge portion 202 about a pitch, yaw and/or roll axis of the handle. For example, an operator may adjust a chopping plane administered to a lens nucleus by rotating knife edge portion 202 about a pitch axis via rotating handle 102 upward (toward a top direction) or downward (toward a bottom direction), the pitch axis being substantially parallel to the equatorial plane of the lens nucleus during chopping. As another example, a chopping direction may be adjusted by rotating knife edge portion 202 about a yaw axis via rotating handle 102 clockwise or counterclockwise relative to a center of a lens nucleus, the yaw axis being substantially perpendicular to the equatorial plane during chopping. As yet another example, an operator may adjust a chopping plane angle by rotating knife edge portion 202 about a roll axis via rotating handle 102 clockwise or counterclockwise about a longitudinal axis of handle 102. It is to be understood that the above pitch, yaw and roll axis descriptions may or may not apply to shaft 104.

Nucleus chopper 100 and its elements may take any size or proportion. For example, a total coaxial length of nucleus chopper 100 may be between 1-4 inches. As another example, a distance between top side 206 and bottom side 208 may be on an order of 0.5-3.0 mm.

Retractor portion 200, knife edge portion 202, cracker areas 204 or any element of multifunctional tip 106 or shaft 104 may be shaped in various ways. For example, FIGS. 3A′, 16A, 16B, 16C, and 16D show various embodiments of a lens nucleus chopping apparatus where a nucleus chopper includes alternative functional tips.

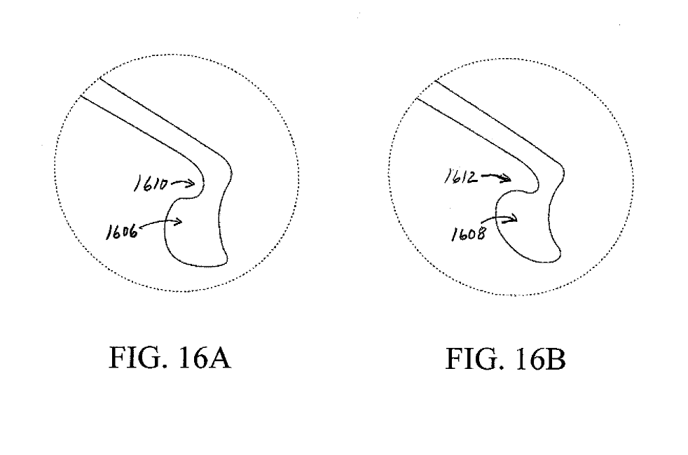

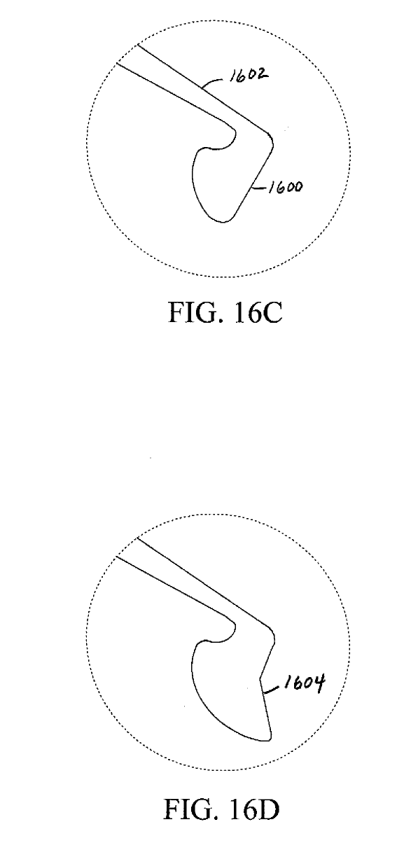

For example, FIG. 3A′ shows knife edge portion 202′ having a curvature with greater concavity compared to knife edge portion 202. FIG. 16C shows knife edge portion 1600 having a substantially straight edge, subtending to shaft 1602. Further, FIG. 16D shows knife edge portion 1604 having a “V” shaped extent. This is to show that an extent of knife edge portion 202 may take various shapes and forms (i.e. crescent, straight, “V” or “C” shaped, and may be configured for particular cataract densities and/or lens nucleus shapes and/or sizes. Knife edge portion 202 may include a sharp edge that tapers from approximately 120 micrometers to a point, where the taper may be on any order of length and angle. As an example knife edge portion 202 may come to a point at one longitudinal side of cracker areas 204, or may come to a point central to both longitudinal sides of cracker areas 204.

FIG. 16A shows cracker areas 1606 having a different shape compared to cracker areas 1608 of FIG. 16B. This is to show that cracker areas of the disclosed nucleus chopper may have various shapes and surface areas. As an example, cracker areas having greater surface area may allow a surgeon to more easily manipulate or crack a nucleus lens. Further, a greater surface area for cracker areas allows the cracker areas to more safely engage a capsular bag surrounding a lens nucleus. Further, FIG. 16A shows that areas of the top side of the disclosed multifunctional tip may have curved and smooth portions. For example, top side 206 of FIG. 2 may include blunt, curved, or smooth portions similar to bottom side 208 of cracker areas 204. This allows the disclosed multifunctional tip to be safer in case top side 206 engages surrounding sensitive tissue.

Further, FIGS. 16A and 16B show alternatively shaped retractor portions 1610 and 1612, respectively, which have different curvatures or shapes compared to retractor portion 200. This retractor portion curvature may vary depending on elasticity or composition or a target capsular bag to be retracted. As such, retractor portion 200 may be configured to retract a capsular bag surrounding a lens nucleus without tearing, rupturing or damaging the capsular bag.

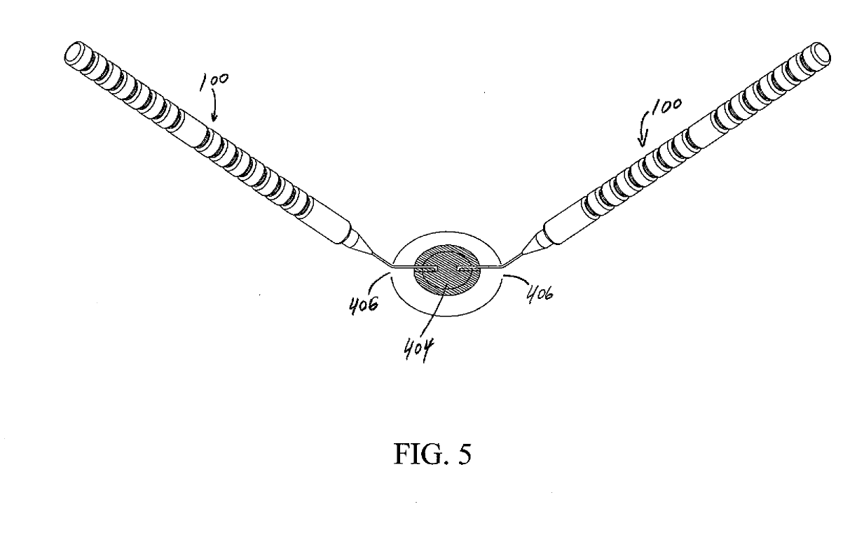

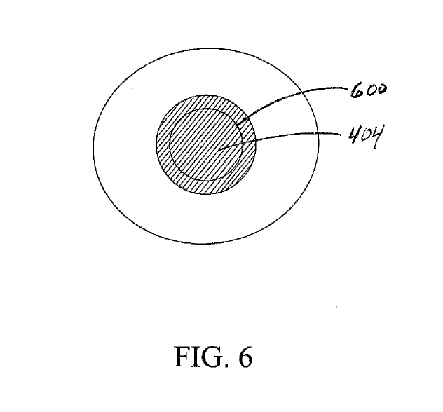

FIGS. 4 and 5 show a pair of nucleus choppers 100. For example, nucleus choppers 100 each include a multifunctional tip 106 as described above. Nucleus choppers 100 are shown in FIGS. 4 and 5 as moving multifunctional tips 106 toward each other in a horizontal manner, across lens nucleus 404. Nucleus choppers 100 may each be inserted through incisions 406 disposed 180 degrees apart relative to a lens nucleus center, for chopping and manipulating lens nucleus 404. For example, incisions 406 may be incised via sclerocorneal tunnel incisions. As such, FIGS. 4 and 5 illustrate incisions 406 made on sclerocorneal area 408. Such sclerocorneal area 408 is also indicated in FIGS. 6-12. Nucleus choppers 100 may each be inserted through incisions with any subtending angle between the incisions without departing from scope of this disclosure. Further incisions 406 may be disposed on any part of an eye. For example, incisions 406 may be made in corneal or scleral portions of an eye, or where corneal and scleral portions substantially meet. Nucleus choppers 100 may be handled during chopping such that each top side of each of multifunctional tips 106 is positioned above an equatorial plane of lens nucleus 404, and each bottom side 208 of each of multifunctional tips 106 is positioned below the equatorial plane. For example, FIG. 15A shows multifunctional tips 106 in such an arrangement for chopping, an equatorial plane indicated by dashed line. To further describe, top side 206 shown in FIG. 2 is preferably held toward an anterior of lens nucleus 404 and bottom side 208 is held toward a posterior of lens nucleus 404. It is to be understood that multifunctional tips 106 may be positioned in various ways relative to the equatorial plane. For example, bottom side 208 may instead be positioned above equatorial plane. Further, it is to be understood that chopping lens nucleus 404 may include only partially incising lens nucleus 404 such that an incision depth does not completely span a thickness of the lens nucleus. Such an incision depth is characterized by a dimension that is perpendicular to the equatorial plane. For example, nucleus choppers 100 may be first incise lens nucleus no deeper than the posterior surface of the lens nucleus before cracking action is applied. This may be beneficial to a surgeon trying to avoid contacting a posterior portion of a capsular bag while chopping action is applied as described above. Further, nucleus choppers 100 allow a surgeon to choose a chopping depth that is not limited by the shaft. It is to be understood that any chopping depth may be applied to lens nucleus 404 via one or both of nucleus choppers 100 before cracking.

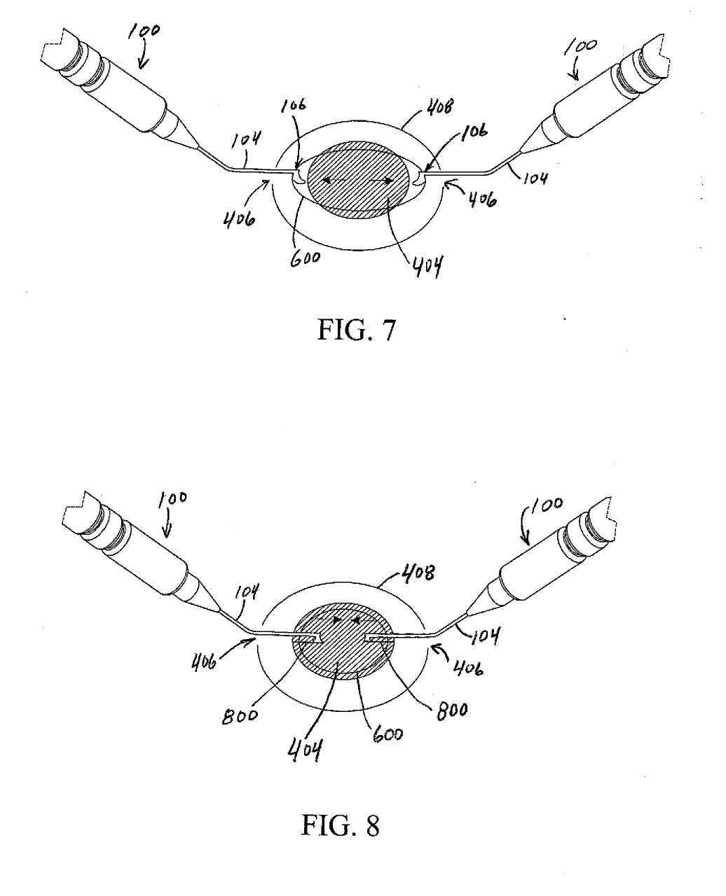

FIGS. 6-12 show example lens nucleus chopping and manipulation in a plan view of lens nucleus 404. FIG. 6 illustrates lens nucleus 404 and capsular bag rim 600 before introduction of nucleus choppers 100 of FIGS. 4 and 5. For example, capsular bag rim 600 may be a rim of a capsular bag that has undergone a capsulorhexis procedure, the capsulorhexis procedure known in the art as described in the background section above. For reference, FIG. 15B shows a cross-sectional view of an eye prior to a capsulorhexis procedure having been performed, where an equatorial plane of lens 404 is perpendicular to the page. FIG. 15B shows various tissues that surround lens nucleus 404, such as zonules 1502, iris 1504, cornea 1506 and capsular bag 1508. It is to be understood that although not shown, lens nucleus 404 is covered by various adjacent layers such as the epinucleus and the cortex. As such, nucleus chopper 100 may chop any of these layers of the lens as described herein. Particularly, nucleus chopper 100 may chop cut any layer that is beneath a capsular bag of an eye. Lens nucleus 404 is shown in FIGS. 6-14 having an equatorial plane substantially parallel to the plane of the page, and perpendicular to one's perspective of the drawing. FIG. 7 shows nucleus choppers 100 symmetrically retracting capsular bag rim 600 with retractor portions 200, such that capsular bag contact points are substantially 180 degrees apart relative to a lens nucleus center, and such that knife edge portions 202 of multifunctional tips 106 are placed at an equatorial portion of lens nucleus 404. This engagement can be seen more closely in FIG. 15A, as the respective knife edge portions 202 of the multifunctional tips 106 engage equatorial portions 1500. After retracting capsular bag rim 600, knife edge portions 202 of the nucleus choppers engage opposite sides of equatorial portions of lens nucleus 404. Incisions 406 of FIGS. 4 and 5 are also shown in FIGS. 7-11.

FIG. 8 shows nucleus choppers 100 being pushed such that multifunctional tips 106 and thus the knife edge portions 202 move toward each other, beginning forming an incision 800 in lens nucleus 404. FIG. 8 also shows capsular bag rim 600 substantially elastically moving back to an original position, such as the position of capsular bag rim 600 shown in FIG. 6. Such elastic properties of a capsular bag is described in more detail in Assia El, Apple DJ, Tsai JC, Lim E S. “The elastic properties of the lens capsule in capsulorhexis.” Am J Ophthalmol. 1991; 111:629-32. It is to be understood that incision 800 may have any depth. For example, incision 800 may completely span a depth from an anterior surface of lens nucleus 404 through to a posterior surface of lens nucleus 404. Alternatively, incision 800 may span a depth from an anterior surface of lens nucleus 404 to any point in between the anterior surface and the posterior surface. It is to be understood that multifunctional tips 106 of nucleus choppers 100 may be operated bimanually to chop or slice a lens nucleus along a same axis of movement such that a target lens nucleus does not rotate when multifunctional tips 106 are pushed toward each other.

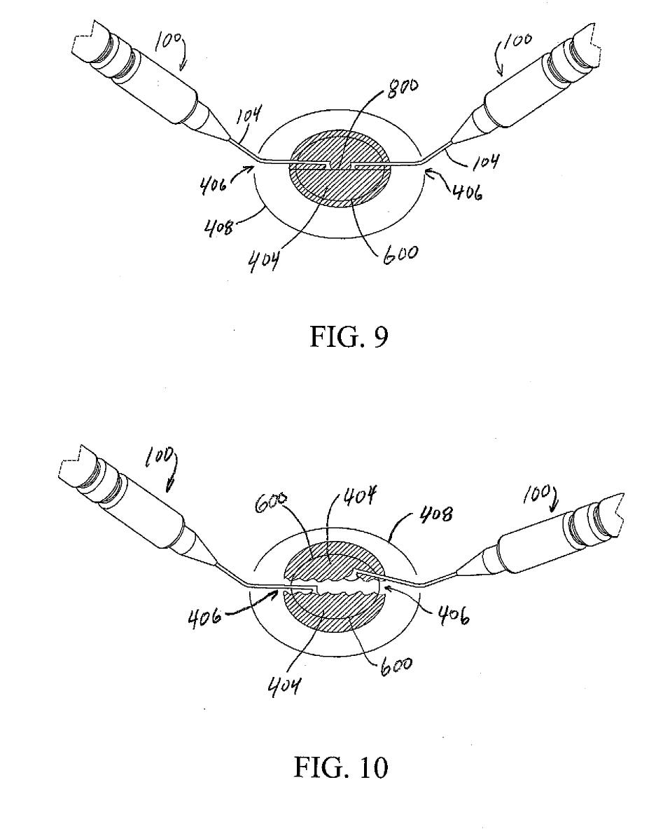

FIG. 9 shows nucleus choppers 100 having substantially incised and or cut lens nucleus 404 such that lens nucleus 404 may be cracked, divided and/or segmented by one or both of multifunctional tips 106. For example, multifunctional tips 106 may move past eachother, for making a proper incision that may be cracked. The arrows indicate the cracking motion or direction.

FIG. 10 shows nucleus choppers 100 having cracked and separated lens nucleus 404 via above described cracking portions. For example, nucleus choppers 100 may crack lens nucleus 404 when an operator applies a force or action to one or both of multifunctional tips 106 in a perpendicular direction to the incision 800 of FIGS. 8 and 9. Such a perpendicular direction is substantially parallel to an equatorial plane of lens nucleus 404, and can be also considered a horizontal direction in accordance with aspects of this disclosure.

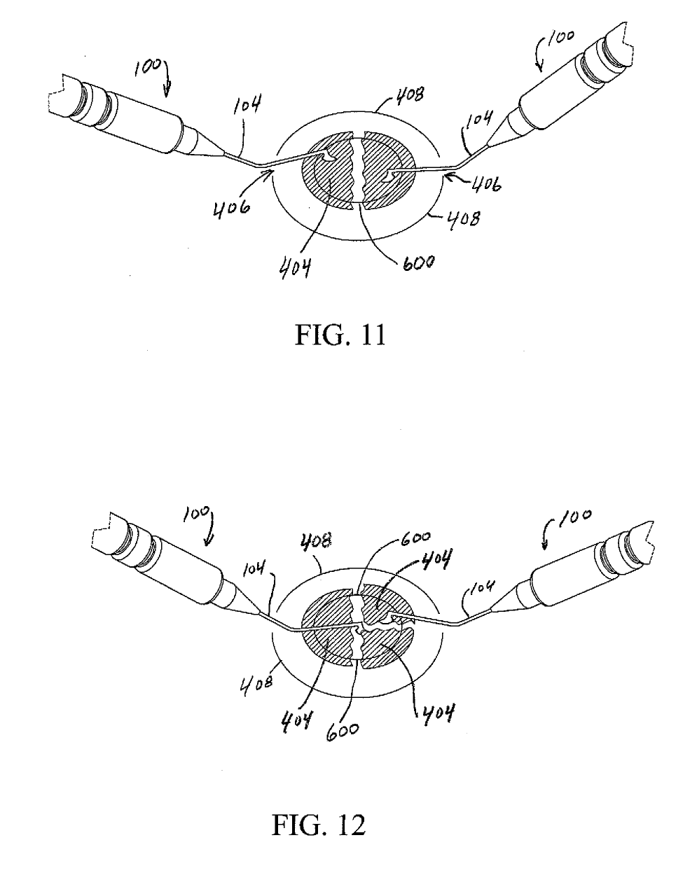

FIG. 11 shows nucleus choppers 100 rotating the incised lens nucleus 404 by a 90 degree angle. This can be accomplished via a surgeon engaging one or both of the above described cracked portions via one or both nucleus choppers 100 to rotate lens nucleus 404.

FIG. 12 shows nucleus choppers 100 incising and cracking lens nucleus 404 once more, in a similar fashion as described with respect to FIGS. 8, 9 and 10. It is to be understood that such a sequence of incisions via nucleus choppers 100 shown in FIGS. 8-12 may be accomplished with or without first retracting capsular bag rim 600.

The above described methods and configurations allow nucleus choppers 100 to chop and/or segment lens nucleus 404 into multiple portions. For example, nucleus choppers 100 may segment lens nucleus 404 into 6-8 separate pieces. FIG. 12 shows lens nucleus 404 being chopped and/or segmented into three pieces.

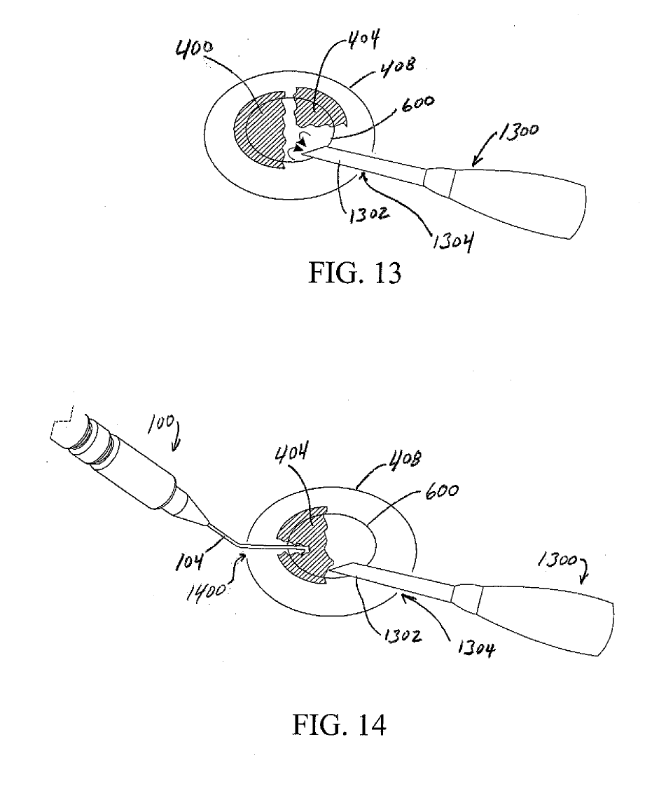

FIG. 13 shows lens nucleus 404 of FIG. 12 after introduction of phacoemulsifier 1300. Phacoemulsifier 1300 includes a phacoemulsifier tip 1302, and is inserted through sclerocorneal area 408 via phaco-incision 1304. FIG. 13 shows a segment of lens nucleus 404 having been phacoemulsified and/or aspirated by phacoemulsifier 1300.

FIG. 14 shows lens nucleus 404 being chopped by nucleus chopper 100 during phacoemulsification. Nucleus chopper 100 is inserted via chopper-incision 1400 similar to incisions 406 of FIGS. 7-12. FIG. 14 shows nucleus chopper 100 chopping lens nucleus 404 via multifunctional tip 106 as described above, while phacoemulsifier 1300 emulsifies and/or aspirates lens nucleus 404. For example, nucleus chopper 100 may be pushed toward phacoemulsifier tip 1302 such that vector forces may substantially cancel, providing a clean chop and/or cut. Nucleus chopper 100 in accordance with aspects of this disclosure may chop and/or cut lens nucleus 404 without lens nucleus 404 rotating and/or moving in undesired directions, because applied vector forces via phacoemulsifier tip 1302 and nucleus chopper 100 may neutralize or cancel in horizontal directions during chopping via knife edge portion 202. This allows a surgeon to chop and/or cut lens nucleus 404 with superior control and effectiveness compared to conventional or traditional nucleus choppers.

As such multifunctional tip 106 is considered multifunctional or multi-faceted, as it is able to cut, slice, chop, retract, rotate, and/or manipulate various tissues during cataract surgery. It is to be understood that lens nucleus chopper 100 and its various elements disclosed herein are not limited for use in cataract surgery, and any appropriate tissue surgery may benefit from aspects of the present invention.

Via the above described configurations, features and/or methods, a surgeon may chop a lens nucleus in improved ways.

For example, when used with phacoemulsification, nucleus chopper 100 enables a surgeon to apply a force to a lens nucleus or fragment via anterior tip side 212, concurrently chopping and pushing and feeding lens nucleus 404 toward phacoemulsifier 1300 via knife edge portion 202. As such, vector forces are substantially horizontal and neutralized providing a safer, more efficient procedure since neutralized opposing vector forces during chopping between a phacoemulsifier and a nucleus chopper may allow cutting or incising without undesired and potentially dangerous movement of a lens nucleus. In other words, nucleus chopper 100 and phacoemulsifier tip 1302 may be pushed toward each other, through lens nucleus 404, on a same axis of movement, since their respective incisions are optimally placed 180 degrees apart relative to the center of the lens nucleus. Even further, features of nucleus chopper 100 enable improved access to areas of lens nucleus 404 when compared to traditional choppers. For example, nucleus chopper 100 can be inserted at chopper-incision 1400 positioned at an opposite side of phacoemulsifier 1300, allowing better engagement coverage since phacoemulsifier 1300 is less in the way of nucleus chopper 100 during bimanual operation, compared to traditional choppers and methods. For example, prior art choppers include a cutting edge on a posterior tip side, near a handle, such that a “dragging” motion must be applied to attempt to chop a lens nucleus. Therefore, prior art choppers are for optimal operation, inserted through an incision that subtends an angle less than 90 degrees to a phacoemulsifier incision, relative to a center of a lens nucleus. As such, during phacoemulsification, a prior art chopper may be blocked by a phacoemulsifier, hindering the traditional chopper from engaging lens nucleus surfaces near a distal side of a phacoemulsifier. Further, since knife edge portion 202 of nucleus chopper 100 is disposed at anterior tip side 212, a number and length of movements required to appropriately engage lens nucleus 404 for chopping is minimized. For example, a traditional chopper requires at least two lengthy motions to engage and attempt to chop a lens nucleus, a first motion to extend the traditional chopper to a central part of the anterior face of the lens nucleus for engagement, then a second pulling motion to attempt to chop. Nucleus chopper 100 can start chopping lens nucleus 404 immediately upon entering an equatorial portion side of lens nucleus 404, as described with respect to FIGS. 4-15, while providing an additional benefit of retracting a capsular bag immediately before chopping action. As an additional benefit, nucleus chopper 100 may be used as an intra-ocular lens (IOL) manipulator including all the benefits and features described above. Even further the present invention is suitable even in case of a shallower anterior chamber. Additionally, when compared to traditional nucleus choppers, the blunt areas near bottom side 208 allow a surgeon to maneuver the multifunctional tip 106 without worrying about damaging sensitive areas surrounding the lens. A surgeon may “slide” bottom side 208 along lens nucleus 404 while maneuvering or positioning multifunctional tip 106. This feature is not available in traditional nucleus choppers since traditional prior art choppers are configured to apply vertical chopping motions. Further, since bottom side 208 includes smooth, blunt surfaces, the multifunctional tip 106 can be operated to chop with little or no vertical motion.

For chopping a lens nucleus without a phacoemulsifier, bimanually operated nucleus choppers 100 allow a surgeon to horizontally chop or slice through a lens nucleus such that horizontal forces are cancelled, reducing or removing net horizontal forces. Traditional choppers do not have this capability, as traditional chopper tips are merely configured to engage an anterior face of a lens nucleus for dragging the tip across the lens nucleus, are not able to retract a capsular bag of an eye, and have a sharp point for providing a vertical plunge-cut (which may puncture a capsular bag or damage tissue). A lens nucleus is composed of fibers that can be seen via a microscope, and a traditional chopper merely “dissects” or “tears” these fibers. The disclosed nucleus choppers 100 may actually “slice” through lens fibers via methods and features described herein.

Nucleus choppers 100 allow an operator to segment, rotate, manipulate, retract, and/or chop lens nucleus 100 and eye tissues in an improved time-efficient manner, solving problems discussed in the background section. For example, nucleus choppers 100 enable lens nucleus 100 to be manually chopped into smaller fragments more safely and efficiently, compared to traditional manual chopping methods. As such, nucleus choppers 100 allow manual extraction of lens nucleus 100 through a smaller, safer incision.

I claim:

1. An eye lens nucleus dividing apparatus comprising:
two instruments, each instrument of said two instruments including:
a handle with a longitudinal end;
a shaft extending from said longitudinal end to a terminal end of said shaft;
a multifunctional tip disposed at said terminal end, said multifunctional tip having a knife edge, a retractor surface opposite said knife edge, and lateral surfaces opposite one another and extending from said knife edge to said retractor surface.

2. The dividing apparatus according to claim 1, wherein said knife edge extends from said terminal end and faces in substantially a same direction as said terminal end.

3. The dividing apparatus according to claim 2, wherein edges of said lateral surfaces define a bottom surface from said knife edge to said retractor surface.

4. The dividing apparatus according to claim 3, wherein said bottom surface and said retractor surface have a smooth finish.

5. The dividing apparatus according to claim 3, wherein said bottom surface and said retractor surface have a rounded cross section.

6. The dividing apparatus according to claim 5, wherein said rounded cross section is defined by a full radius between said lateral surfaces.

7. The dividing apparatus according to claim 6, wherein said rounded cross section is defined by a two corner radii at corners of said lateral surfaces.

8. The dividing apparatus according to claim 3, wherein said bottom surface is defined by a curve.

9. The dividing apparatus according to claim 1, wherein said knife edge has a concave extent.

10. The dividing apparatus according to claim 1, wherein each retractor surface is configured to retract a capsular bag of an eye lens.

11. A method of splitting a lens nucleus of an eye, comprising:
providing two surgical apparatus each including a respective multifunctional tip, the multifunctional tip having a retractor surface, a knife edge, and lateral surfaces extending from the knife edge to the retractor surface;
retracting a capsular bag of the lens nucleus with the retractor surfaces;
pushing the knife edge of at least one apparatus of the two apparatus through the lens nucleus towards the knife edge of a remaining apparatus of the two apparatus for chopping the lens nucleus; and
applying action to one or two of the apparatus for cracking the lens nucleus with respective lateral surfaces of the apparatus.

12. The method of claim 11, wherein subsequent to retracting the capsular bag, respective knife edges of each apparatus are placed on respective equatorial portions of the lens nucleus for chopping.

13. The method of claim 11, wherein the pushing step includes pushing the knife edge of the at least one apparatus toward the knife edge of the remaining apparatus from opposite sides of the lens nucleus until the knife edges substantially meet for allowing the lens nucleus to be separated into two parts with the lateral surfaces.

14. The method of claim 11, wherein cracking the lens nucleus includes applying action to one or both of the surgical apparatus in a direction parallel to an equatorial plane of the lens nucleus.

15. A method of splitting a lens nucleus of an eye during phacoemulsification, comprising:
providing an instrument that includes a handle with a longitudinal end, a shaft extending from the longitudinal end to a terminal end of the shaft, a multifunctional tip disposed at the terminal end, the multifunctional tip having a knife edge, a retractor surface opposite the knife edge, and lateral surfaces opposite one another and extending from the knife edge to the retractor surface;
emulsifying the lens nucleus with a phacoemulsifier; and
chopping the lens nucleus with the knife edge.

16. The method of claim 15, further comprising:
feeding the lens nucleus to the phacoemulsifier with the knife edge.

17. The method of claim 15, wherein chopping the lens nucleus includes pushing the knife edge through the lens nucleus and toward the phacoemulsifier.

18. The method of claim 16, wherein feeding the lens nucleus to the phacoemulsifier tip includes pushing lens nucleus particles toward the phacoemulsifier tip such that the terminal end of the shaft moves toward the phacoemulsifier tip.

19. The method of claim 18, wherein pushing the lens nucleus particles toward the phacoemulsifier tip includes chopping the lens nucleus with the knife edge.

20. The method of claim 15, further comprising, prior to chopping the nucleus, retracting a capsular bag rim of the lens nucleus with the retractor surface.
